(19) United States
(12) Patent Application Publication
(10) Pub. No.: US 2004/0143153 A1
(43) Pub. Date: Jul. 22, 2004

(54) Devices and methods for manipulation of organ tissue

(76) Inventor: James S. Sharrow, Bloomington, MN (US)

Correspondence Address: SHUMAKER & SIEFFERT, P. A., 8425 SEASONS PARKWAY, SUITE 105, ST. PAUL, MN, 55125, US

(21) Appl. No.: 10/347,847

(22) Filed: Jan. 17, 2003

(51) Int. Cl. A61F 2/00

(52) U.S. Cl. 600/37

(57) ABSTRACT

In general, the invention is directed to techniques for lifting and positioning an organ, such as a heart, with two or more manipulating devices. One manipulating device serves as a lifting member, bearing a substantial amount of the weight of the organ, and another manipulating device serves as a positioning member, orienting or stabilizing the organ in a desired position. The manipulating devices, which may be vacuum-assisted, are coupled to one another with an adjustable structural connector, which secures the manipulating devices in a substantially fixed position relative to one another.

### TECHNICAL FIELD

The invention relates to devices capable of providing adherence to organs of the body for purposes of medical diagnosis and treatment. More particularly, the invention relates to devices capable of adhering to, holding, moving, stabilizing or immobilizing an organ.

### BACKGROUND

In many areas of surgical practice, it may be desirable to manipulate an internal organ without causing damage to the organ. In some circumstances, the surgeon may wish to turn, lift or otherwise reorient the organ so that surgery may be performed upon it. In other circumstances, the surgeon may simply want to move the organ out of the way. In still other cases, the surgeon may wish to hold the organ, or a portion of it, immobile so that it will not move during the surgical procedure.

Unfortunately, many organs are slippery and are difficult to manipulate. Holding an organ with the hands may be undesirable because of the slipperiness of the organ. Moreover, the surgeon's hands ordinarily cannot hold the organ and perform the procedure at the same time. The hands of an assistant may be bulky, becoming an obstacle to the surgeon. Also, manual support of an organ over an extended period of time can be difficult due to fatigue. Holding an organ with an instrument may damage the organ, especially if the organ is unduly squeezed, pinched or stretched. Holding an organ improperly may also adversely affect the functioning of the organ.

The heart is an organ that may be more effectively treated if it can be manipulated. Many forms of heart manipulation may be useful, including moving the heart within the chest and holding it in place. Some forms of heart disease, such as blockages of coronary vessels, may best be treated through procedures performed during open-heart surgery. During open-heart surgery, the patient is typically placed in the supine position. The surgeon performs a median sternotomy, incising and opening the patient's chest. Thereafter, the surgeon may employ a rib-spreader to spread the rib cage apart, and may incise the pericardial sac to obtain access to the heart. For some forms of open-heart surgery, the patient is placed on cardiopulmonary bypass (CPB) and the patient's heart is arrested. Stopping the patient's heart is a frequently chosen procedure, as many coronary procedures are difficult to perform if the heart continues to beat. CPB entails trauma to the patient, with attendant side effects and risks. An alternative to CPB involves operating on the heart while the heart continues to beat.

Once the surgeon has access to the heart, it may be necessary to lift the heart from the chest or turn it to obtain access to a particular region of interest. Such manipulations are often difficult tasks. The heart is a slippery organ, and it is a challenging task to grip it with a gloved hand or an instrument without causing damage to the heart. Held improperly, the heart may suffer ischemia, hematoma or other trauma. The heart may also suffer a loss of hemodynamic function, and as a result may not pump blood properly or efficiently. Held insecurely, the heart may drop back into the chest, which may cause trauma to the heart and may interfere with the progress of the operation.

The problems associated with heart manipulation are greatly multiplied when the heart is beating. Beating causes translational motion of the heart in three dimensions. In addition, the wringing action of heart activity cause the heart to twist when beating. These motions of the heart make it difficult to lift the heart, move it and hold it in place.

In a coronary bypass operation, for example, the surgeon may need to manipulate the heart. The affected coronary artery may not be accessible without turning or lifting of the heart. Once the heart has been lifted or turned, the surgeon may need to secure the heart in a substantially fixed position.

### SUMMARY

In general, the invention is directed to surgical techniques for lifting and positioning an organ, such as a heart, with two or more manipulating devices. One manipulating device serves as a lifting member, bearing a substantial amount of the weight of the organ, and another manipulating device serves as a positioning member, serving to orient or stabilize the organ in a desired position. The positioning member may also bear some of the load of the organ.

The manipulating devices are coupled to one another with an adjustable structural connector, which secures the manipulating devices in a substantially fixed position relative to one another. The manipulating devices may be coupled directly to the structural connector, but in a typical embodiment, the manipulating devices may be coupled to the structural connector via a supporting structure. For example, the lifting and positioning devices may be coupled to support shafts, and the support shafts may be secured in a fixed position with an adjustable joint.

The manipulating devices may adhere to the organ with the assistance of vacuum pressure. A vacuum source may supply vacuum pressure to one or more manipulating devices via one or more vacuum tubes. The vacuum pressure may cause at least a portion of the manipulating devices to deform and substantially form a seal against the surface of the tissue of the organ. In some embodiments of the invention, one or more vacuum tubes may also serve as support shafts, and the lifting and positioning devices may be placed in a substantially fixed position relative to one another by securing the vacuum tubes in a fixed position with an a structural connector. In other embodiments of the invention, a vacuum tube may play little or no part in lifting or positioning the organ.

The manipulating devices may be of many different types, and the invention is not limited to any particular type or types. Manipulating devices may have a one-piece or multi-piece construction, for example, and may be of a variety of shapes and sizes. The invention also encompasses manipulating devices that are not vacuum-assisted.

The invention accommodates the use of non-rigid couplings, which grant some limited freedom of motion to the manipulating devices. Non-rigid couplings are especially useful when the lifting and positioning members are applied to a heart, because non-rigid couplings accommodate the natural motions of the heart. This limited freedom of motion helps preserve the hemodynamic functions of the heart, making the patient less likely to suffer from circulatory problems during surgery. Examples of non-rigid couplings include swivels, flexible stems or nipples, and positioning joints having a limited range of motion.

In one embodiment, the invention is directed to an apparatus comprising a first and a second manipulating device, each having a surface to contact an organ, and a structural connector that adjustably holds the second manipulating device in a position relative to the first manipulating device. The first, manipulating device is configured to bear a substantial amount of the weight of the organ, and the second manipulating device is configured to substantially position the organ.

The structural connector may include a first housing and a second housing that may be in either an engaged position or a disengaged position. The housings are positionable relative to one another when in the disengaged position, and resist motion relative to one another when in the engaged position. A securing member such as a spring-loaded connector or threaded connecting pin and knob may force the housings into the engaged position or the disengaged position.

The manipulating devices may be vacuum-assisted. Each manipulating device may be served by an independent vacuum source. In some embodiments of the invention, however, a single vacuum source may serve two or more manipulating devices, with the assistance of a valve element. In the event that vacuum pressure to one manipulating device is compromised, the valve element helps maintain vacuum pressure in the other manipulating device.

In another embodiment, the invention is directed to an apparatus comprising a first manipulating device having a first surface to contact an organ and defining a first chamber, and a second manipulating device having a second surface to contact the organ and defining a second chamber. The apparatus also includes a first vacuum tube in fluid communication with the first chamber and a second vacuum tube in fluid communication with the second chamber. The apparatus further includes a structural connector that includes a securing member that secures the position of the first vacuum tube relative to the second vacuum tube.

In a further embodiment, the invention is directed to an apparatus comprising a first manipulating device, a second manipulating device, a first support shaft coupled to the first manipulating device, a second support shaft coupled to the second manipulating device, and a structural connector. The structural connector includes a securing member that secures the position of the support shafts relative to on another. One or more support shafts may be, but need not be, hollow and serve as vacuum tubes.

In an additional embodiment, the invention is directed to a method for manipulating an organ. The method comprises engaging a first manipulating device with an apex of a heart to define a first chamber, engaging a second manipulating device with the heart at a site other than the apex to define a second chamber, and applying vacuum pressure to the first and second chambers. The method also includes substantially supporting the weight of the heart with the first manipulating device and positioning the heart with the second manipulating device. The method may further include securing the first and second manipulating devices in a substantially fixed position relative to one another.

In another embodiment, the invention is directed to a method comprising engaging a first manipulating device with an organ and engaging a second manipulating device with the organ. The first manipulating device and the second manipulating device are each coupled to respective support shafts. The method further includes orienting one support shaft into a position relative to the other support shaft, and securing the support shafts into the position. The method also comprises substantially supporting the weight of the organ with the first manipulating device and positioning the organ with the second manipulating device.

In a further embodiment, the invention is directed to a method comprising engaging a first manipulating device to an organ, lifting the organ with the first manipulating device, engaging a second manipulating device to the organ, and positioning the organ with the second manipulating device.

The invention can provide one or more advantages. For example, the organ may be held in place more securely with multiple manipulating devices than with a single manipulating device. Moreover, the organ can be manipulated with the lifting and positioning members so that the surgeon may have access to a desired region of the organ. When the invention is used with a heart, the heart may be lifted and turned without causing trauma and without stopping the heart.

In addition, various embodiments include features that may be advantageous in particular circumstances or with some configurations of apparatus. The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective view of an organ supporting apparatus, in conjunction with a heart.

FIG. 2 is a side view of the exemplary joint shown in FIG. 1.

FIG. 3 is a perspective view of an alternate embodiment of an organ supporting apparatus, in conjunction with a heart.

FIG. 4 is an exploded view of the exemplary joint shown in FIG. 3.

FIG. 5 is a an exploded perspective view of a lifting support shaft housing and a positioning support shaft housing shown in FIG. 4.

FIG. 6 is an exploded perspective view of a positioning joint shown in FIG. 3.

FIG. 7 is a perspective view of an alternate embodiment of an organ supporting apparatus, in conjunction with a heart.

FIG. 8 is cross-sectional side view of one exemplary embodiment of a bifurcated valve shown in FIG. 7.

FIG. 9 is cross-sectional side view of the bifurcated valve shown in FIG. 8 showing the valve in operation.

FIG. 10 is cross-sectional side view of another exemplary embodiment of a bifurcated valve shown in FIG. 7.

FIG. 11 is cross-sectional side view of the bifurcated valve shown in FIG. 8 showing the valve in operation.

FIG. 12 is a perspective view of an exemplary bleed vent.

FIG. 13 is a cross-sectional side view of the bleed vent shown in FIG. 12.

FIG. 14 is a cross-sectional side view of the bleed vent shown in FIG. 12, showing the bleed vent in operation.

### DETAILED DESCRIPTION

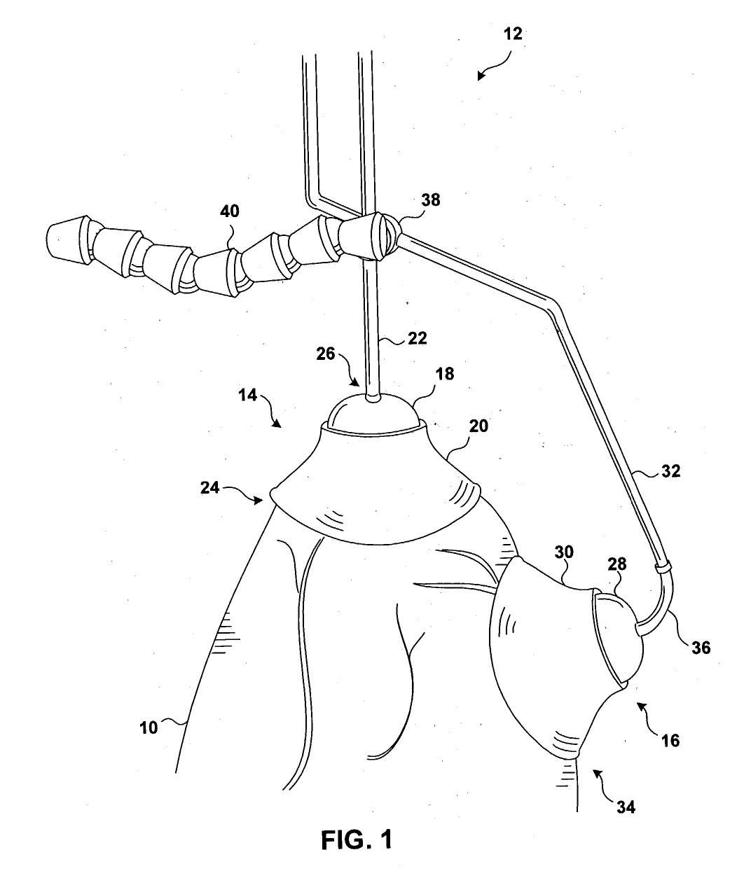

FIG. 1 shows a human heart 10 supported by an exemplary organ supporting apparatus 12. Organ supporting apparatus 12 comprises at least two manipulating devices 14 and 16. Manipulating devices 14, 16 are in contact with the surface of heart 10. In one embodiment, manipulating device 14 may include a cup-like member 18, which defines a general size and shape of the manipulating device 14. Cup-like member 18 defines a generally circular structure suitable for forming a cup-like shape. Cup-like member 18 may also provide a firm structure by which manipulating device 14 may be securely gripped by a surgeon or by an instrument.

Cup-like member 18 may be formed from many materials, including thermoplastic such as polycarbonate, ABS, polysulfone, polyester and polyurethane, and including corrosion-resistant metals such as titanium, and including rigid and semi-rigid elastomers such as silicone rubber, natural rubber, synthetic rubber, and polyurethane. Cup-like member 18 may have a semi-rigid structure that may be somewhat compliant, but generally resistant to deformation. For example, cup-like member 18 may be formed from a silicone elastomer of Shore A 30 to 70 durometer.

Cup-like member 18 may be coupled to a skirt-like member 20. Skirt-like member 20 may be formed from a substantially compliant material, such as a silicone gel, hydrogel or closed cell foam. Skirt-like member 20 may be, for example, molded from silicone elastomer of Shore A 5 to 10 durometer. Skirt-like member 20 generally deforms upon contact with tissue. In this way, cup-like member 18 imparts structural integrity to manipulating device 14, and skirt-like member 20 conforms to the general shape of the organ, thereby facilitating a seal interface with the tissue of heart 10. The material forming skirt-like member 20 may be substantially compliant, making skirt-like member 20 less likely to cause trauma. The material forming skirt-like member 20 may also include a tacky substance that promotes adhesion to the surface of the tissue, such as biocompatible adhesive or silicone gel.

The interior wall of cup-like member 18 and skirt-like member 20 define a chamber (not shown in FIG. 1). Vacuum tube 22, coupled to cup-like member 18, provides fluid communication between the chamber of manipulating device 14 and a vacuum source (not shown in FIG. 1). The vacuum source may supply vacuum pressure by way of vacuum tube 22, causing at least a portion of manipulating device 14 to deform and substantially form a seal against the surface of the tissue of heart 10. Vacuum pressure may be supplied by a number of vacuum sources, such as by a syringe or a pump.

In FIG. 1, manipulating device 14 has been placed in contact with the apex 24 of heart 10, such that the chamber defined by cup-like member 18 and skirt-like member 20 receive apex 24. Manipulating device 14 has been affixed to apex 24 by the application of vacuum pressure via vacuum tube 22. The surgeon may mount manipulating device 14 on apex 24 while heart 10 reclines in its natural position in the chest of the patient. By application of vacuum pressure via vacuum tube 22, manipulating device 14 adheres to apex 24. A tacky surface of skirt-like member 20 may also aid adhesion. The surgeon may lift heart 10 by manipulating device 14 or vacuum tube 22, elevating apex 24 to the position shown in FIG. 1. By lifting heart 10 in this manner, the surgeon may obtain access to a particular region of interest.

Manipulating device 14 is configured to bear a substantial amount of the weight of heart 10. For example, manipulating device 14 is sized and shaped to have a substantial contact with apex 24 of heart 10, such that heart 10 can be lifted by manipulating device 14 affixed to apex 24. Manipulating device 14 is further constructed to bear the load of the lifted heart 10 securely, without dropping heart 10.

Manipulating device 14 may be coupled to vacuum tube 22 by a non-rigid coupling that accommodates motion of heart 10. In FIG. 1, manipulating device 14 is coupled to vacuum tube 22 by a swivel connection 26, which is one example of a non-rigid coupling. Swivel connection 26 may allow the surgeon to position vacuum tube 22 relative to manipulating device 14 in a convenient and/or expedient fashion. Swivel connection 26 may further assist the surgeon in mounting manipulating device 14 to apex 24 and may also accommodate the natural motion of heart 10. Heart 10 may expand, contract, twist and move in translational fashion with each beat while manipulating device 14 is affixed to apex 24. Swivel connection 26 moves with apex 24, giving heart 10 some freedom of motion. The freedom of motion helps preserve the hemodynamic functions of heart 10. As a result, the patient is less likely to suffer from circulatory problems during surgery.

Manipulating device 16 is similar to manipulating device 14, and includes a cup-like member 28 and skirt-like member 30 that define a chamber. Vacuum tube 32 may supply vacuum pressure from a vacuum source (not shown in FIG. 1), causing at least a portion of manipulating device 16 to deform and substantially form a seal against the surface of the tissue of heart 10. Manipulating device 16 is affixed to a side 34 of heart 10. Cup-like member 28 and skirt-like member 30 may be, but need not be, constructed like cup-like member 18 and skirt-like member 20 of manipulating device 14. As will be shown below, manipulating devices 14, 16 are merely illustrative of manipulating devices. The invention encompasses manipulating devices of a variety of shapes, sizes, and properties.

The vacuum source supplying vacuum pressure to manipulating device 14 may be, but need not be, the same vacuum source supplying vacuum pressure to manipulating device 16. There may be advantages to supplying vacuum pressure to manipulating devices 14, 16 from independent vacuum sources. Independent vacuum sources offer a margin of safety, as one manipulating device may continue to hold the organ when the other manipulating device loses its seal with the tissue or loses its vacuum supply.

Manipulating device 16 may be coupled to vacuum tube 32 by a non-rigid coupling. In FIG. 1, manipulating device 16 is coupled to vacuum tube 32 by a flexible stem 36 that serves as a flexible joint between manipulating device 16 and vacuum tube 32. Flexible stem 36 allows the surgeon to substantially fix the position of manipulating device 16 relative to manipulating device 14 in a convenient or expedient fashion, but also accommodates the natural motion of heart 10.

Swivel connection 26 and flexible stem 36 are examples of connections between vacuum tubes 22, 32 and manipulating devices 14, 16. Other forms of non-rigid couplings may also be used, and additional examples of couplings will be described below. The invention encompasses all forms of non-rigid and rigid couplings.

In the typical application shown in FIG. 1, vacuum tube 22 may be constructed from materials that are flexible and that also are strong in tension, such as silicone rubber. Strength in tension is important in the application shown in FIG. 1 because vacuum tube 20 and manipulating device 14 are configured to substantially support the weight of heart 10. Vacuum tube 22 may also be constructed from rigid or semi-rigid materials, such as titanium or rigid polymers.

Vacuum tube 32, which supplies vacuum pressure to manipulating device 16, may likewise be constructed from rigid or semi-rigid materials. In contrast to vacuum tube 20 and manipulating device 14, which bear a substantial amount of the weight of heart 10, vacuum tube 32 and manipulating device 16 substantially position or stabilize heart 10. Rigidity of vacuum tube 32 may therefore be a desirable quality, because rigidity helps maintain the orientation of heart 10 in a desired position. In addition, when vacuum tube 32 is rigid, vacuum tube 32 and manipulating device 16 can assist vacuum tube 22 and manipulating device 14 in bearing some of the load of heart 10.

Manipulating devices 14, 16 and vacuum tubes 22, 32 cooperate to provide two points of stability. Manipulating device 14 acts as a lifting member, bearing a substantial amount of the load of heart 10. Manipulating device 16 acts as a positioning member, serving to orient heart 10 in a desired position and perhaps bearing some of the load of heart 10. Using manipulating devices 14, 16 and vacuum tubes 22, 32, the surgeon can lift, rotate and orient heart 10 to a desired position.

Vacuum tube 22 is secured to vacuum tube 32 with a structural connector in the form of a joint 38. Joint 38 is an adjustable device that allows the lifting and positioning members, i.e., manipulating devices 14 and 16, to be oriented in a desired position relative to one another. In the application shown in FIG. 1, joint 38 holds manipulating device 14 in a desired position relative to manipulating device 16 by holding vacuum tube 22 in a desired position relative to vacuum tube 32. When vacuum tubes 22, 32 are oriented as desired, joint 38 may be locked, fixing the position of vacuum tube 22 relative to vacuum tube 32. Examples of adjustable and lockable joints will be described in more detail below.

Joint 38 contributes to the stability of organ supporting apparatus 12. In particular, joint 38 contributes to the stability of manipulating devices 14 and 16 relative to one another and to heart 10. Joint 38 prevents vacuum tubes 22, 32 from separating and moving freely. In this way, joint 38 promotes cooperation to provide the two points of stability afforded by manipulating devices 14, 16 and vacuum tubes 22, 32.

Supporting arm 40 supports organ supporting apparatus 12. Supporting arm 40 may be affixed to a relatively immovable object, such as a rib spreader (not shown) or an operating table (not shown). In FIG. 1, supporting arm 40 is shown coupled to joint 38. This arrangement is for purposes of illustration, and supporting arm 40 may be coupled to organ supporting apparatus 12 in other ways as well. Supporting arm 40 be coupled to vacuum tubes 22, 32 above or below joint 38, for example. Supporting arm 40 need not be a segmented articulable arm as shown in FIG. 1, but may be, for example, a solid rod or bar.

In FIG. 1, the load of heart 10 is principally supported by manipulating device 14, vacuum tube 22, joint 38 and supporting arm 40. Heart 10 is principally stabilized by manipulating device 16, vacuum tube 32, joint 38 and supporting arm 40.

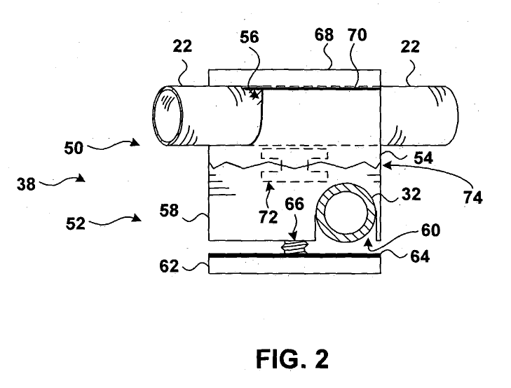

FIG. 2 shows an exemplary embodiment of joint 38. Joint 38 comprises housings 50, 52, which hold vacuum tubes 22, 32. Housing 50 includes a hub 54, which has a channel 56 that receives vacuum tube 22. Similarly, housing 52 includes a hub 58 with a channel 60 that receives vacuum tube 32. Housing 52 includes a cap 62 and a gasket 64, which are coupled to hub 58 with a screw 66. Cap 62 and gasket 64 are slightly unscrewed and separated from hub 58 for clarity. While cap 62 and gasket 64 are unscrewed, vacuum tube 32 is free to slide in channel 60. In some embodiments of joint 38, cap 62 and gasket 64 may be further separated from hub 58, permitting vacuum tube 32 to be removed completely from channel 60.

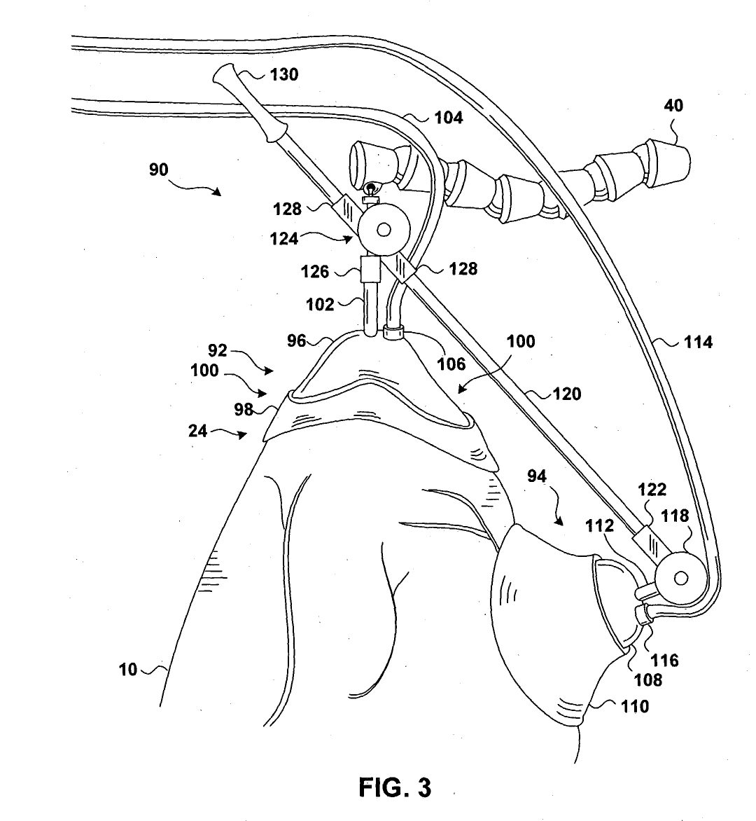

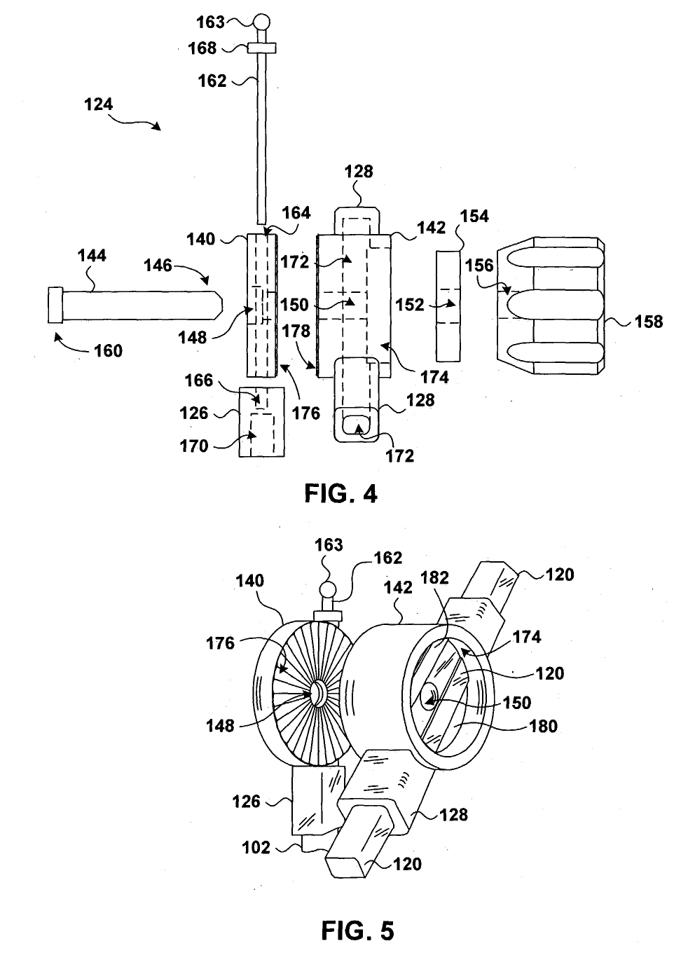

Similarly, housing 50 includes a cap 68 and a gasket 70, which are coupled to hub 54 with a screw (not shown). Cap 68 and gasket 70 are shown screwed into hub 54, securing vacuum tube 22 in place. Gasket 70 deforms against vacuum tube 22 to frictionally stop vacuum tube 22 from sliding in channel 56. Cap 68 with gasket 70 represent one embodiment of a securing member that secures vacuum tube 22 in place, relative to joint 38. Other securing members may include clips, clasps, or a washer and knob such those as described below in connection with FIG. 4.

By securing vacuum tubes 22 and 32 in place, joint 38 substantially fixes the position of vacuum tubes 22 and 32 relative to one another. In this way, joint 38 substantially fixes the position of manipulating device 14 relative to manipulating device 16.

Hubs 54, 58 and caps 62, 68 may be formed from a rigid material such as metal or plastic. Gaskets 64, 70 may be formed from a pliable material such as polyurethane, silicone or rubber.

Hubs 54, 58 are coupled to one another with a hub connector 72. Hub connector 72 may be formed from a rigid material such as metal or plastic. Hub connector 72 may be spring-loaded to pull hubs 54, 58 toward one another. In addition, hubs 54, 58 may include mating surfaces 72, which, when engaged, resist rotation of hubs 50, 52 relative to one another. An operator such as a surgeon can set the position of housings 50, 52 relative to one another by pulling housings 50, 52 apart, thereby overcoming the pull of hub connector 72 and causing mating surfaces 72 to disengage.

When mating surfaces 72 are disengaged, housings 50, 52 can be rotated relative to one another. Once housings 50, 52 are in a desired orientation, the operator may release housings 50, 52. Hub connector 72 pulls housings 50, 52 toward one another and mating surfaces 72 engage once again. In this manner, the orientation of vacuum tubes 22, 32 may be fixed by adjusting joint 38. Hub connector 72 may include a locking mechanism (not shown in FIG. 2) such as a toggle clamp that, when engaged, prevents housings 50, 52 from being pulled apart and prevents mating surfaces 72 from disengaging.

In some embodiments, mating surfaces 72 may include a pattern of lines, grooves, protrusions, indentations and the like. A pattern of radiating ridges similar to that found on poker chips, for example, may suffice. In other embodiments, mating surfaces 72 may be formed from a material having a high coefficient of friction, or may include a texture that resists the rotation of housings 50, 52 relative to one another.

Joint 38 may further include a mounting device (not shown) that mounts or affixes joint 38 to supporting arm 40. A typical mounting device may be coupled to either housing 50, 52 or to both housings 50, 52. A mounting device may allow freedom of motion of joint 38 relative to support arm 40 in some respects and may restrict freedom of motion in other respects A mounting device may also allow freedom of motion in one configuration, and be securable in another configuration that restricts freedom of motion.

Joint 38 is an exemplary embodiment of a structural connector. Other embodiments of a structural connector may adjustably hold manipulating devices 14, 16 in a position relative to one another. The invention is not limited to a structural connector embodied as a joint, nor is the invention limited to the particular structural connectors shown in the figures.

FIG. 3 shows heart 10 supported by an organ supporting apparatus 90 in accordance with an alternative embodiment of the invention. Organ supporting apparatus 90 comprises at least two manipulating devices 92 and 94, both of which are in contact with the surface of heart 10. Manipulating devices 92, 94 cooperate to provide two points of stability with respect to heart 10. Manipulating device 92 acts as a lifting member, bearing a substantial portion of the load of heart 10, and manipulating device 94 acts as a positioning member, serving to orient heart 10 in a desired position and perhaps bearing some of the load of heart 10.

Manipulating device 92 may include a shell member 96, which defines a general size and shape of the manipulating device 92. Shell member 96 serves many of the same functions as cup-like member 18 shown in FIG. 1, except that shell member 96 may be specially shaped for application to apex 24. Shell member 96 need not be cup-shaped, and need not be symmetrical. Shell member 96 may be coupled to a skirt-like member 98. Skirt-like member 98, which may be formed in from the same materials as skirt-like members 20 and 30 in FIG. 1, may be any shape. In FIG. 3, shell member 96 and skirt-like member 98 define a plurality of projections 100 that extend radially outward from the center of shell member 96 and conform to the irregular shape of heart 10. Projections 100 may be, but need not be, of uniform size, shape or spacing. Shell member 96 and skirt-like member 98 may define a chamber that receives apex 24 of heart 10.

Like manipulating device 14 in FIG. 1, manipulating device 92 is configured to bear a substantial amount of the weight of heart 10. Unlike manipulating device 14, which is supported by vacuum tube 22, manipulating device 92 is supported by a lifting support shaft 102. Lifting support shaft 102 may be, for example, a flexible shaft that accommodates the natural motion of heart 10. Lifting support shaft 102 does not supply vacuum pressure to manipulating device 92. Instead, vacuum tube 104, coupled to manipulating device 92 via vacuum port 106, supplies vacuum pressure to manipulating device 92, causing skirt-like member 98 to deform and substantially form a seal against the surface of the tissue of heart 10. Vacuum port 106 provides fluid communication between the chamber of manipulating device 92 and a vacuum source (not shown). Unlike vacuum tube 22 shown in FIG. 1, vacuum tube 104 bears little, if any, of the weight of heart 10.

Manipulating device 94 is similar to manipulating devices 14, 16 in that manipulating device 94 includes a cup-like member 108 and a skirt-like member 110. Manipulating device 94 is different from manipulating device 14 and manipulating device 16, however, in that manipulating device 94 is supported by a support member 112 separate from the supply of vacuum pressure. Vacuum tube 114, coupled to manipulating device 94 via vacuum port 116, supplies vacuum pressure to manipulating device 94. Like lifting support shaft 102, support member 112 may be a non-rigid coupling that provides some freedom of motion to heart 10.

Support member 112 is coupled to a positioning joint 118, which in turn is coupled to a positioning support shaft 120. Positioning support shaft 120 may bear some of the load of heart 10, but positioning support shaft 120 principally positions or stabilizes heart 10. Positioning support shaft 120 may be formed from any of a number of rigid materials, including plastics and metals.

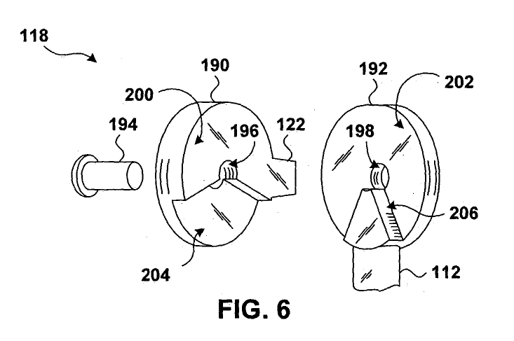

Positioning support shaft 120 may be mated to a receptacle 122 on positioning joint 118 that receives shaft 120. In one embodiment, the range of motion of positioning joint 118 may be restricted, keeping skirt-like member 110 oriented toward heart 10 and preventing manipulating device 94 from swinging away from heart 10 in the event manipulating device 94 loses its seal with the tissue or loses its vacuum supply. An example of this embodiment will be described below in connection with FIG. 6.

Lifting support shaft 102 is secured to positioning support shaft 120 with a structural connector such as joint 124. Joint 124 may include a receptacle 126 that receives lifting support shaft 102, and a sleeve 128 that receives positioning support shaft 120. Joint 124 is an adjustable device that allows lifting support shaft 102 to be oriented into a desired position relative to positioning support shaft 120, thereby substantially fixing the position of manipulating device 92 relative to manipulating device 94.

Positioning support shaft 120 may include a handle 130, which may be used to orient positioning support shaft 120. As will be described below, when positioning support shaft 120 is oriented as desired, positioning support shaft 120 may be locked in place. Joint 124 promotes cooperation between manipulating devices 92 and 94 by limiting the freedom of movement of manipulating devices 92 and 94 relative to one another, thereby providing two points of stability.

Heart 10, manipulating devices 92, 94, support shafts 102, 120 and joint 124 may be supported by a supporting arm 40. Supporting arm 40 may be affixed to a relatively immovable object, and may be coupled to organ supporting apparatus 90 at any of several sites. In a typical application, supporting arm 40 may be affixed to joint 124 with a mounting device. An exemplary mounting device is described below.

FIG. 4 is an exploded side view of joint 124. Joint 124 includes a lifting support shaft housing 140 coupled to a positioning support shaft housing 142 with a connecting pin 144. Connecting pin 144 may include threads (not shown) along the length or may include threads (not shown) near tail end 146. When joint 124 is assembled, connecting pin 144 passes through a bore 148 in lifting support shaft housing 140 and through a bore 150 in positioning support shaft housing 142. Connecting pin 144 further passes through a bore 152 in washer 154 and into a receiving recess 156 in knob 158. Receiving recess 156 may include threads (not shown) that cooperate with threads near tail end 146 of connecting pin 144. Head end 160 of connecting pin 144 seats in lifting support shaft housing 140. Head end 160 may be bonded to lifting support shaft housing 140, or may include a rough texture or a locking shape that impedes rotation of head end 160 relative to lifting support shaft housing 140.

Pivot pin 162 passes through an aperture 164 in lifting support shaft housing 140 and is coupled to a receiving recess 166 in receptacle 126. Aperture 164 may be offset from center such that aperture 164 does not intersect bore 148. Pivot pin 162 may be coupled to receptacle 126 by crimping, welding, screwing, adhesive bonding, and the like. Head assembly 168 prevents pivot pin 162 from passing completely through aperture 164. Receptacle includes a recess 170 for receiving lifting support shaft 102 (not shown in FIG. 4), which may be coupled to recess 170 by crimping, welding, screwing, adhesive bonding, and the like.

When joint 124 is assembled, pivot pin 162 is free to rotate in aperture 164. Receptacle 126 and lifting support shaft 102 are therefore free to rotate relative to lifting support shaft housing 140. As heart 10 twists, lifting support shaft 102, receptacle 126 and pivot pin 162 may rotate relative to lifting support shaft housing 140. In this way, joint 124 is another embodiment of a non-rigid coupling that accommodates motion of heart 10.

Joint 124 may include a mounting device such as a ball 163 on pivot pin 162 that facilitates the connection of pivot pin 162 to support arm 40 (shown in FIG. 3). Ball 163 may mate to a receiving structure such as a socket in support arm 40. The socket may be shaped to prevent ball 163 from pulling free of the socket under an applied load, while allowing ball 163 freedom to rotate relative to support arm 40. With a mounting device such as ball 163, pivot pin 162 may rotate relative to support arm 40, and consequently joint 124 may rotate relative to support arm 40, further accommodating the motion of heart 10.

Joint 124 may combine the construction of lifting support shaft 102, receptacle 126 and pivot pin 162. In particular, lifting support shaft housing 140 may include a sleeve (not shown) having an aperture that receives lifting support shaft 102. Although lifting support shaft 102 may have some freedom to slide in the aperture, lifting support shaft 102 may also include a stop (not shown) that prevents lifting support shaft 102 from passing completely through the sleeve. Also, the lifting support shaft 102 and the aperture in the sleeve may be cylindrical, allowing lifting support shaft 102 to rotate in the aperture as heart 10 twists.

Positioning support shaft housing 142 includes sleeve 128, which includes an aperture 172 that slidably receives positioning support shaft 120 (not shown in FIG. 4). Aperture 172 may be offset from center such that aperture 172 does not intersect bore 150. Aperture 172 may be shaped to impede rotation of positioning support shaft 120 inside aperture 172. For example, FIG. 4 depicts aperture 172 as substantially rectangular, and aperture 172 would thereby impede rotation of a substantially rectangular positioning support shaft 120 inside aperture 172.

Positioning support shaft housing 142 further includes large recess 174, which receives washer 154. Washer 154 may be formed from a flexible material such as rubber or silicone. When seated in large recess 174, washer 154 may bear against positioning support shaft 120, frictionally holding positioning support shaft 120 in place and preventing positioning support shaft 120 from sliding inside aperture 172.

Knob 158 may bear against washer 154. When knob 158 is twisted, the mating threads on connecting pin 144 and recess 156 may cause tail end 146 of connecting pin 144 to move deeper into knob 158, thereby pushing knob 158 against washer 154 and pushing washer 154 against positioning support shaft 120. Knob 158 and washer 154 represent one embodiment of a securing member that secures positioning support shaft 120 in place. Other embodiments of securing members may also be employed.

Twisting knob 158 also pushes positioning support shaft housing 142 against lifting support shaft housing 140. Mating faces 176, 178 of lifting support shaft housing 140 and positioning support shaft housing 142, respectively, may thereby be forced together. As will be shown below, mating faces 176, 178 may include a pattern of lines, grooves, protrusions, indentations and the like that secure lifting support shaft housing 140 against positioning support shaft housing 142.

In this way, knob 158 may fix the position of lifting support shaft housing 140 relative to positioning support shaft housing 142, and may further fix the position of positioning support shaft 120 relative to positioning support shaft housing 142. The invention is not limited to the particular design of joint 124 presented in the figures. Instead of knob 158, for example, the joint may include toggle clamp. In this variation, the position of the components may be fixed by pushing a cam toggle rather than by twisting a knob.

FIG. 5 is a perspective partially exploded view of lifting support shaft housing 140 and positioning support shaft housing 142. Receptacle 126 is coupled to lifting support shaft housing 140 by pivot pin 162. In addition, positioning support shaft 120 is threaded through aperture 172 in sleeve 128. Positioning support shaft 120 is visible when positioning support shaft 120 enters and exits sleeve 128, and is also visible inside large recess 174. When washer 154 is seated in large recess 174, washer 154 may bear against positioning support shaft 120, frictionally holding positioning support shaft 120 in place.

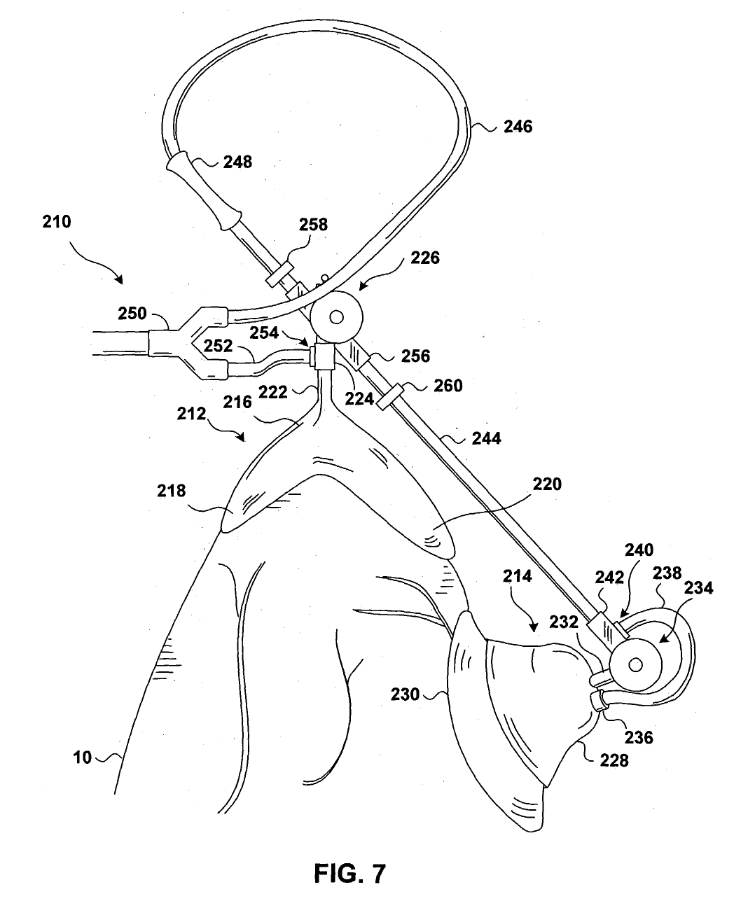

In FIG. 5, positioning support shaft 120 in sleeve 128 is depicted as a solid bar. As will be described below in connection with FIG. 7, sleeve 128 may also accommodate a positioning support shaft that is substantially rigid and hollow. A hollow positioning support shaft may supply vacuum pressure to a manipulating device.

The inner surface 180 of large recess 174 may include a raised portion 182. When washer 154 is seated in large recess 174, washer 154 may bear against positioning support shaft 120 and against raised portion 182. Positioning support shaft 120 and raised portion 182 cooperate to prevent washer 154 from becoming skewed upon engagement with positioning support shaft 120, thereby seating washer 154 substantially parallel to inner surface 180.

FIG. 5 further shows a pattern of radiating ridges on mating surface 176 of lifting support shaft housing 140. A complementary pattern may be on mating surface 178 (not shown in FIG. 5) of positioning support shaft housing 142. When mating surfaces 176, 178 bear against each other, mating surfaces 176, 178 engage and resist rotation of housings 140, 142 relative to one another. Mating surfaces 176, 178 may be forced into an engaged position by rotation of knob 158 in one direction, and loosened into a disengaged position by rotation of knob 158 in the other direction.

FIG. 6 is a perspective exploded view of positioning joint 118. Positioning joint 118 includes hubs 190, 192, coupled by a pivot connector 194 through bores 196, 198. Hubs 190, 192 include smooth facing surfaces 200, 202. When positioning joint 118 is assembled, facing surfaces 200, 202 come in contact, but do not lock together. Hub 192 may rotate to a degree around pivot connector 194, relative to hub 190.

The amount of rotation is limited by one or more restricting structures. In the embodiment shown in FIG. 6, hub 190 includes a wedge-shaped notch 204, which receives a wedge-shaped protrusion 206 on hub 192. The angle of wedge-shaped notch 204 is larger than the angle of wedge-shaped protrusion 206. In one embodiment, the angle of wedge-shaped notch 204 is one hundred twenty degrees and the angle of wedge-shaped protrusion 206 is sixty degrees. When positioning joint 118 is assembled, wedge-shaped protrusion 206 may move inside wedge-shaped notch 204, but by no more than sixty degrees. In this way, the rotational freedom of hubs 190, 192 relative to one another may be restricted.

Because the range of rotational motion of hubs 190, 192 relative to one another may be restricted, manipulating device 94 (shown in FIG. 3) may be oriented toward heart 10. Skirt-like member 110 may be easily brought into engagement with heart 10. In addition, the reduced range of rotational motion reduces the risk that manipulating device 94 may swing away from heart 10 in the event manipulating device 94 loses its seal with the tissue or loses its vacuum supply.

FIG. 7 shows heart 10 supported by an organ supporting apparatus 210 in accordance with an alternative embodiment of the invention. Organ supporting apparatus 210 comprises at least two manipulating devices 212 and 214, both of which are in contact with the surface of heart 10. Manipulating devices 212 acts as a lifting member and manipulating device 214 acts as a positioning member.

Manipulating device 212 has a one-piece construction, comprising a central body 216 and one or more projections that extend outward from central body 216. In FIG. 7, projections 218 and 220 are visible, but other projections may be hidden behind heart 10. Projections 218 and 220 may conform to the irregular shape of heart 10. Projections 218, 220 may be, but need not be, of uniform size, shape or spacing. Central body 216 and projections may define a chamber that receives apex 24 of heart 10.

Manipulating device 212 may also include a nipple 222. Nipple 222 may serve as a conduit for vacuum pressure to manipulating device 212. Nipple 222 may also serve as a support shaft for manipulating device 212. Nipple 222 is coupled to receptacle 224 of joint 226 by any coupling technique, such as crimping or adhesive bonding. In this manner, nipple 222 supports central body 216 like a lifting support shaft, and joint 226 bears the load of manipulating device 212 and a substantial amount of the weight of heart 10 via nipple 222.

In addition, nipple 222 may be flexible, and may twist with respect to joint 226. Nipple 226 may be another embodiment of a non-rigid coupling that accommodates motion of heart 10. Receptacle 224 may also have freedom to rotate relative to joint 226, and may also accommodate motion of heart 10.

Manipulating device 212 may sized and shaped to bear a substantial amount of the weight of heart 10, and may be constructed from one or more materials that exhibit levels of flexibility and compliance. The materials may, for example, include elastomers such as silicone, natural rubber, synthetic rubber, and polyurethane, and more compliant materials, such as silicone gel, hydrogel, or closed cell foam. Manipulating device 212 may comprise a one-piece cast of silicone of sufficiently low durometer to permit deployment and sealing over the curved surfaces of heart 10, while maintaining sufficient structural integrity when subjected to vacuum pressure and the load of heart 10. The durometer of the silicone may, for example, be within the range from 5 to 50 Shore A.

Manipulating device 214 comprises a rigid outer shell 228 coupled to a compliant inner shell 230. Outer shell 228 provides structural integrity to manipulating device 214, and may be formed from metallic or polymeric materials, such as silicone elastomers in the range of Shore A 30 to 75 durometer. Inner shell 230 forms a seal with the tissue in a manner similar to skirt-like members described above, and may be formed from polymeric materials, such as silicone elastomers of approximately Shore A 5 to 50 durometer. Outer shell 228 or inner shell 230 or both shells may define a chamber that may receive tissue of heart 10.

Manipulating device 214 is coupled by a support member 232 to positioning joint 234, and receives vacuum pressure via vacuum port 236. Flexible vacuum tube 238 couples vacuum port 236 on manipulating device 214 to a vacuum port 240 on receptacle 242 of positioning joint 234. Receptacle 242 receives positioning support shaft 244.

Positioning support shaft 244 is substantially rigid and hollow, and supplies vacuum pressure to manipulating device 214, which cooperate to position or stabilize heart 10. Flexible vacuum tube 246 supplies vacuum pressure to positioning support shaft 244. Handle 248 may be used to orient positioning support shaft 244 and may further serve as a port for coupling vacuum tube 246 to positioning support shaft 244. Vacuum tube 246 receives vacuum pressure via bifurcated valve 250, which will be descried in more detail below. Bifurcated valve 250 receives vacuum pressure from a vacuum source (not shown). In this way, bifurcated valve 250 supplies vacuum pressure to manipulating device 214.

Bifurcated valve 250 also supplies vacuum pressure to manipulating device 212. In the embodiment shown in FIG. 7, a flexible vacuum tube 252 conveys vacuum pressure from bifurcated valve 250 to a port 254 in receptacle 224 of joint 226, and receptacle 224 conveys vacuum pressure to nipple 222 of manipulating device 212.

Apart from port 254, joint 226 may be a structural connector similar to joint 124 described above in connection with FIGS. 3, 4 and 5. In the embodiment depicted in FIG. 7, joint 226 includes a sleeve 256 similar to sleeve 128 of joint 124. Likewise, positioning joint 234 may be similar to positioning joint 118 described above in connection with FIGS. 3 and 6.

Travel stops 258, 260 may be coupled to positioning support shaft 244. Travel stops 258, 260 are unable to pass through the aperture of sleeve 256, limiting the extent to which positioning support shaft 244 can slide inside sleeve 256, thereby reducing the risk that positioning support shaft 244 will be moved so as to dislodge vacuum tube 246 or handle 248. Dislodging vacuum tube 246 or handle 248 may result in an undesirable loss of vacuum pressure. Travel stops may be, for example, O-rings formed from an elastomeric material, or may be bands affixed to or integrally formed with positioning support shaft 244.

Heart 10, manipulating devices 212, 214 and joint 226 may be supported by a supporting arm (not shown).

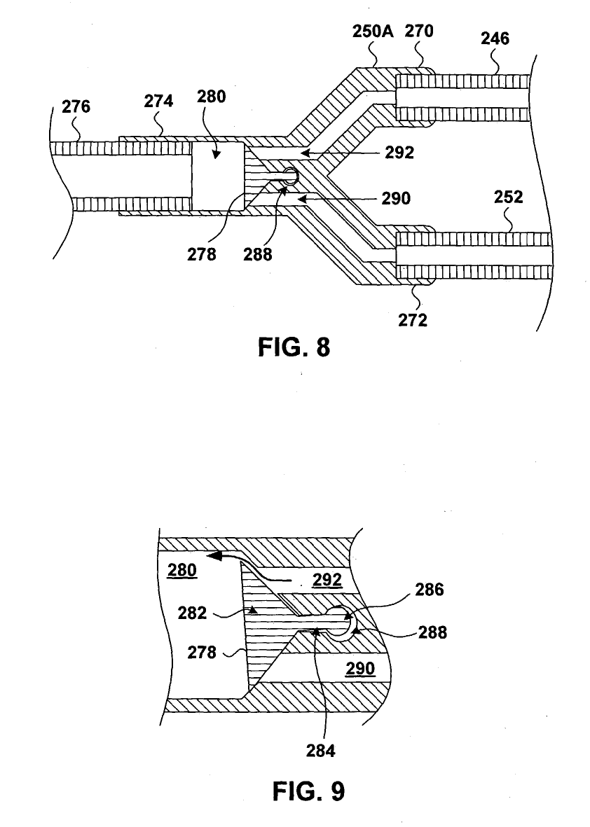

FIG. 8 shows a cross-sectional side view of an embodiment of a bifurcated valve 250A. Bifurcated valve 250A includes fittings 270, 272 that receive vacuum tubes 246, 252. For purposes of illustration, it will be assumed that vacuum tube 246 supplies vacuum pressure to a manipulating device that serves as a positioning member, and vacuum tube 252 supplies vacuum pressure to a manipulating device that serves as a lifting member.

Bifurcated valve 250A also includes a fitting 274 that receives a vacuum tube 276. Vacuum tube 276 may be coupled to a vacuum source (not shown). Fitting 274 may include a stop such as a ridge or a block (not shown) to prevent vacuum tube 276 from coming in contact with valve element 278, which will be described below. A space 280 separates vacuum tube 276 from valve element 278, which gives valve element 278 some freedom to move, as will be described below.

A vacuum supplied by a single vacuum source may supply vacuum pressure to vacuum tubes 246, 252, which in turn supply vacuum pressure to distinct manipulating devices. Should the seal of one manipulating device rupture, it is desirable that the vacuum to the other manipulating device be protected. Valve element 278 protects the vacuum in one manipulating device in the event the seal of the other manipulating device should fail.

FIG. 9 provides a close-up view of valve element 278 and illustrates an exemplary motion to maintain a vacuum. Valve element 278 comprises a main vane body 282, a stem 284 and an anchor 286. Anchor 286 is seated in mating cavity 288. Valve element 278 may be shaped substantially like a prism. Mating cavity 288 is larger than stem 284 and anchor 286, so valve element 278 has some freedom of motion. Mating cavity 288 is not large enough, however, to release anchor 286.

In the example shown in FIG. 9, it is assumed that the manipulating device that serves as a positioning member has lost a seal with the tissue, but the manipulating device that serves as a lifting member has not. Accordingly, passage 290, which supplies vacuum pressure to the lifting member, remains at a pressure below ambient pressure. A pressure gradient develops in passage 292, however, because the positioning member has lost the seal. The pressure gradient forces main vane body 282 away from passage 292, and simultaneously deflects main vane body 282 to occlude passage 290. As main vane body 282 deflects due to the pressure difference between passage 292 and space 280, stem 284 and anchor 286 pivot in mating cavity 288.

In the event the seal of the lifting member is compromised but the seal of the positioning member is maintained, main vane body 282 may deflect away from passage 290 and simultaneously occlude passage 292. In the event there is no seal for either the lifting member or the positioning member, pressure gradients will force main vane body 282 away from both passages 290, 292.

The components of bifurcated valve 250A may be made of any a number of materials, such as metal or plastic. In one embodiment, the components may be molded from silicone and may have varying degrees of hardness. The frame of bifurcated valve 250A may be made from silicone with a hardness of approximately Shore A 80 durometer. Vacuum tubes 246, 252, 276, by contrast, may have a hardness of approximately Shore A 30 to 50 durometer, and thus be more flexible than bifurcated valve 250A. Valve element 278 may also be molded from silicone, and may have a hardness of approximately Shore A 80 durometer. In a variation of this embodiment, valve element 278 may comprise a core having a hardness of approximately Shore A 80 durometer, and soft sealing surfaces (not shown) that reduce leakage when main vane body 282 occludes passage 290 or passage 292. The soft sealing surfaces may have a hardness of approximately Shore A 10 durometer.

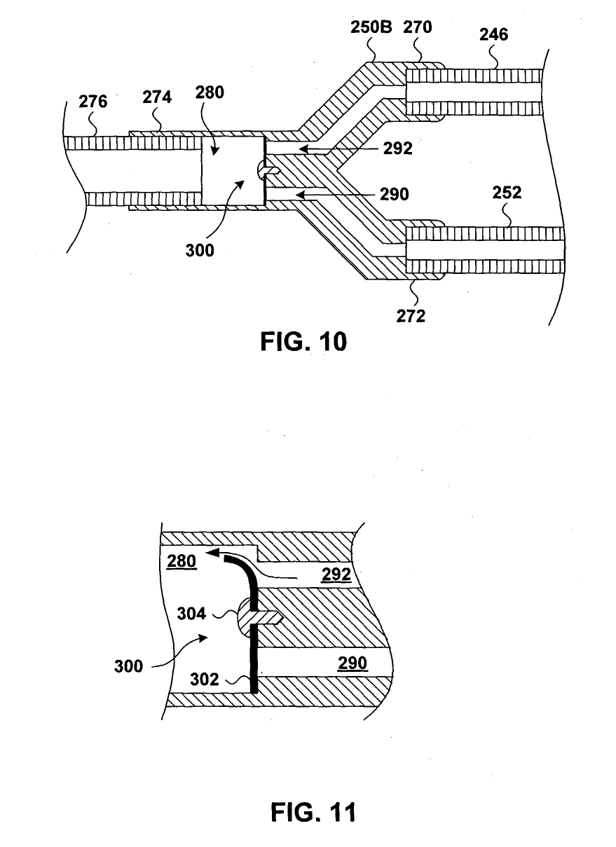

FIG. 10 shows a cross-sectional side view of an another embodiment of a bifurcated valve 250B. Bifurcated valve 250B, like bifurcated valve 250A, includes fittings 270, 272 that receive vacuum tubes 246, 252. Bifurcated valve 250B also includes a fitting 274 that receives a vacuum tube 276.

Bifurcated valve 250B includes valve element 300, shown in more detail in FIG. 11. Valve element 300 comprises a flexible flap 302 held in place with a pin 304. As shown in FIG. 11, flap 302 may deflect due to a pressure difference between passage 292 and space 280, while simultaneously occluding passage 290. In this way, the seal of the positioning member may be maintained even if the seal of the lifting member is compromised. In similar fashion, the seal of the lifting member may be maintained even if the seal of the positioning member is compromised. In the event there is no seal for either the lifting member or the positioning member, pressure gradients will force flap 302 away from both passages 290, 292.

Flap 302 may be molded from a pliable material such as silicone having a hardness of approximately Shore A 10 durometer. The hardness of flap 302 may vary depending upon the thickness of flap 302. Although depicted in FIG. 11 as a single piece, flap 302 may be supplanted with separate flaps for passages 290, 292.

Manipulating devices 212, 214 or any vessel that supplies vacuum pressure to a manipulating device may include one or more bleed vents (not shown in FIGS. 7-11). Bleed vents may be opened when the surgeon desires to disengage a manipulating device from the organ by disrupting the vacuum that holds the manipulating device against the organ.

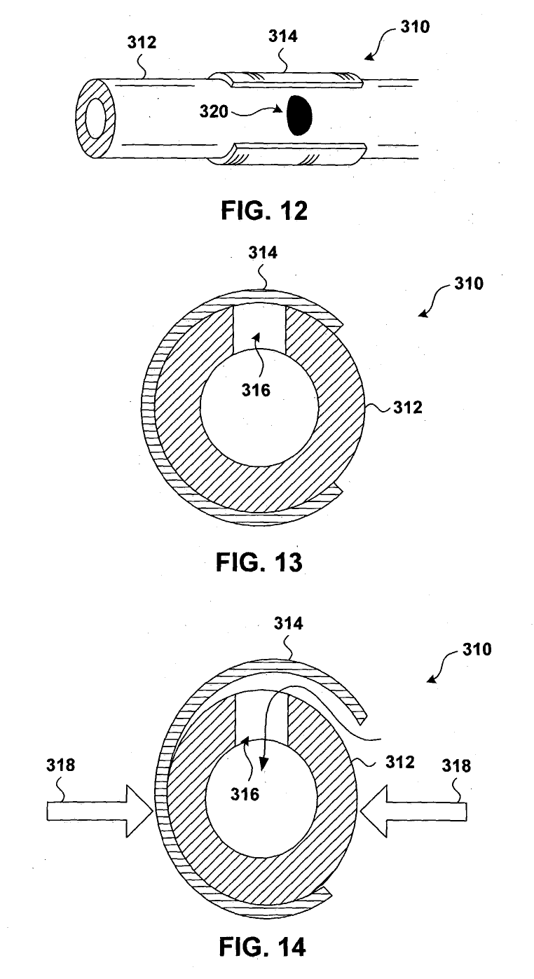

An exemplary bleed vent 310 is depicted in FIGS. 12-14. A flexible vacuum tube 312 may include a vent cover 314. In ordinary use, vent cover 314 occludes a port 316. Port 316 may be any shape, such as circular or rectangular. When bleed vent 310 is squeezed as indicated by arrows 318 in FIG. 14, vent cover 314 may separate from port 316, allowing air to enter vacuum tube 312. Medical personnel may open port 316 by squeezing bleed vent 310 with fingers, or with a medical instrument such as a clamp. Vacuum tube 312 and vent cover 314 may include visible indicators such as dot 320 that show where to squeeze bleed vent 310 to open port 316. Vent cover 314 may be secured to vacuum tube 312 with adhesive so that vent cover 314 is free to occlude or separate from port 316 but is not free to slide along or disengage from vacuum tube 312.

Vacuum tube 312 may be formed from a flexible material such as silicone and have a hardness of approximately Shore A 30 to 50 durometer. Vent cover 314 likewise may be formed from a flexible material such as silicone. Vent cover 314 and have a hardness of approximately Shore A 30 to 50 durometer.

Bleed vent 310 is one example of many possible designs for bleed vents, and the invention is not limited to the particular bleed vent shown. Bleed vents need not be limited to incorporation in flexible vacuum tubes, but may be included on a substantially rigid tube such as handle 248 or positioning support shaft 244 shown in FIG. 7. Bleed vents may also be included on one or more manipulating devices.

By operating a bleed vent, medical personnel may relieve the vacuum pressure for one manipulating device without affecting the vacuum pressure of the other manipulating device. When the vacuum pressure is relieved in a manipulating device, the manipulating device may be disengaged from the organ. The manipulating device may further be repositioned, and reengaged to the organ. When used with a bifurcated valve such as valve 250A or 250B, relieving vacuum pressure in one manipulating device need not affect vacuum pressure in any other manipulating device.

The invention can provide one or more advantages. For example, the organ may be held in place more securely with multiple manipulating devices than with a single manipulating device. Moreover, the organ can be manipulated with the lifting and positioning members so that the surgeon may have access to a desired region of the organ. When the invention is used with a heart, the heart may be lifted and turned without causing trauma and without stopping the heart. Various embodiments of the invention grant the heart limited freedom of movement so that the hemodynamic functions of the heart are preserved. As a result, the patient is less likely to suffer from circulatory problems during surgery.

Various embodiments of the invention have been described. These embodiments are illustrative of the practice of the invention. Many of the elements of the described embodiments may be applied with other embodiments. As demonstrated by FIGS. 1, 3 and 7, different types of manipulating devices may be used as lifting and positioning members. A manipulating device depicted herein as a lifting member may be used as a positioning member, and vice versa. In addition, lifting and positioning members may include a tacky substance on one or more surfaces to promote adhesion to the surface of the tissue.

In some embodiments, vacuum tubes may be flexible, rigid, or part flexible and part rigid. Load-bearing supports, such as support shafts, may be hollow and include a passage to supply vacuum pressure to the manipulating devices. Vacuum pressure may also be supplied independent of the load-bearing supports. Vacuum pressure may be also be supplied through a structural connector.

The invention may also be practiced with one or more manipulating devices that do not use a vacuum source. A manipulating device may adhere to an organ by a tacky substance, for example, or may adhere like a suction cup, not requiring a constant source of vacuum pressure. A manipulating device or vacuum tube may further include a valve that, when open, allows vacuum pressure to be supplied to the manipulating device, and when closed, maintains the vacuum pressure in the chamber of the manipulating device by blocking air entry into the chamber. A manipulating device may also include a hydraulic chamber filled with a hydraulic fluid, and adhesion between the manipulating device and the organ may be accomplished by controlling the shape or fluid content of the hydraulic chamber.

Although the manipulating devices described herein include compliant surfaces that contact the organ, the invention encompasses manipulating devices that include non-compliant surfaces as well. Compliant surfaces are generally more desirable, however, because compliant surfaces are usually less prone to causing trauma.

Several embodiments of non-rigid couplings have been described. The couplings may be included in a structural connector such as a joint or in a manipulating device or in a structure that couples a structural connector to a manipulating device. The invention is not limited to any particular form of non-rigid coupling, and includes embodiments that do not comprise a non-rigid coupling.

In addition, some embodiments may include a single structure that may perform multiple functions. Nipple 222, for example, serves as a support shaft, a conduit for delivering vacuum pressure, and a non-rigid coupling. The invention encompasses embodiments in which a single structure plays more than one role.

Several embodiments of structural connectors have been described. The invention is not limited to any particular form of structural connector, and other embodiments of structural connectors may be employed to hold a positioning member in a desired position relative to a lifting member. Structural connectors may include one or more securing members to hold positioning and lifting members in position frictionally, or may use other techniques to secure positioning and lifting members in position.

Various modifications may be made without departing from the scope of the claims. For example, there may be multiple lifting members and/or multiple positioning members. In some applications, multiple manipulating devices may be in contact with the surface of an organ, with each manipulating device bearing part of the load of the organ. Similarly, multiple manipulating devices may simultaneously position the organ. Although the embodiments described herein are shown with reference to a heart, the invention may be applied to other organs as well. These and other embodiments are within the scope of the following claims.

1. An apparatus comprising:
a first manipulating device having a first surface to contact an organ;
a second manipulating device having a second surface to contact the organ; and
a structural connector that adjustably holds the second manipulating device in a position relative to the first manipulating device;
wherein the first manipulating device is configured to bear a substantial amount of the weight of the organ, and
wherein the second manipulating device is configured to substantially position the organ.

2. The apparatus of claim 1, further comprising a non-rigid coupling that couples the structural connector to the first manipulating device.

3. The apparatus of claim 2, wherein the non-rigid coupling comprises at least one of a swivel connection and a flexible stem.

4. The apparatus of claim 1, wherein the first manipulating device comprises a flexible nipple coupled to the structural connector.

5. The apparatus of claim 1, further comprising a support shaft that couples the structural connector to the first manipulating device.

6. The apparatus of claim 5, wherein the support shaft is hollow.

7. The apparatus of claim 6, wherein the first manipulating device comprises a vacuum port, and wherein the support shaft is coupled to the vacuum port.

8. The apparatus of claim 1, further comprising a rigid support shaft that couples the structural connector to the second manipulating device.

9. The apparatus of claim 8, wherein the support shaft is hollow.

10. The apparatus of claim 8, wherein the structural connector comprises a sleeve with an aperture that slidably receives the support shaft.

11. The apparatus of claim 8, wherein the structural connector comprises a securing member that secures the support shaft in position relative to the structural connector.

12. The apparatus of claim 1, wherein the second manipulating device defines a chamber, the apparatus further comprising a vacuum port in fluid communication with the chamber.

13. The apparatus of claim 12, further comprising a vacuum tube coupled to the vacuum port.

14. The apparatus of claim 13, wherein the vacuum tube includes a bleed vent.

15. The apparatus of claim 1, wherein at least one of the first surface and the second surface includes a material that promotes adhesion to the organ.

16. The apparatus of claim 1, wherein the structural connector comprises:
a first housing; and
a second housing;
wherein the first housing and the second housing may be in one of an engaged position and a disengaged position, are positionable relative to one another when in the disengaged position and resist motion relative to one another when in the engaged position.

17. The apparatus of claim 16, wherein at least one of the first and second housings comprises a channel to receive a vacuum tube.

18. The apparatus of claim 16, wherein at least one of the first and second housings comprises an aperture to receive a support shaft.

19. The apparatus of claim 16, wherein the structural connector further comprises a spring-loaded connector that forces the first housing and the second housing into the engaged position.

20. The apparatus of claim 16, wherein the structural connector further comprises a threaded connecting pin and a knob that receives the connecting pin, wherein the first housing and the second housing may be forced into one of the engaged position and the disengaged position by twisting the knob.

21. The apparatus of claim 16, wherein the first housing includes a first mating surface and the second housing includes a second mating surface, wherein the first mating surface contacts the second mating surface when the first housing and the second housing are in the engaged position, and wherein the first and second mating surfaces resist motion relative to one another when the first housing and the second housing are in the engaged position.

22. The apparatus of claim 1, wherein the structural connector comprises a mounting device configured to couple to a supporting arm.

23. The apparatus of claim 1, wherein the first manipulating defines a first chamber and the second manipulating defines a second chamber, the apparatus further comprising:
a first vacuum tube in fluid communication with the first chamber; and
a second vacuum tube in fluid communication with the second chamber.

24. The apparatus of claim 23, further comprising a valve that couples the first vacuum tube and the second vacuum tube to a source of vacuum pressure.

25. The apparatus of claim 24, further comprising a valve element that occludes fluid flow in one of the first and second vacuum tubes when a pressure gradient develops in the other of the first and second vacuum tubes.

26. The apparatus of claim 25, wherein the valve element comprises one of a vane body and a flap.

27. An apparatus comprising:
a first manipulating device having a first surface to contact an organ and defining a first chamber;
a second manipulating device having a second surface to contact the organ and defining a second chamber;
a first vacuum tube in fluid communication with the first chamber;
a second vacuum tube in fluid communication with the second chamber; and
a structural connector that receives the first and second vacuum tubes, the structural connector including a securing member that secures the position of the first vacuum tube relative to the second vacuum tube.

28. The apparatus of claim 27, further comprising a non-rigid coupling that couples the first vacuum tube to the first manipulating device.

29. The apparatus of claim 27, further comprising a non-rigid coupling that couples the second vacuum tube to the second manipulating device.

30. The apparatus of claim 27, wherein the first manipulating device is configured to bear a substantial amount of the weight of the organ and the second manipulating device is configured to substantially position the organ.

31. The apparatus of claim 30, wherein the second vacuum tube is substantially rigid.

32. The apparatus of claim 27, wherein the structural connector comprises a first housing that receives the first vacuum tube and a second housing that receives the second vacuum tube.

33. The apparatus of claim 32, wherein the first housing and the second housing may be in one of an engaged position and a disengaged position, are positionable relative to one another when in the disengaged position and resist motion relative to one another when in the engaged position.

34. An apparatus comprising:
a first manipulating device having a first surface to contact an organ;
a second manipulating device having a second surface to contact the organ;
a first support shaft coupled to the first manipulating device;
a second support shaft coupled to the second manipulating device; and
a structural connector that receives the first and second support shafts, the structural connector including a securing member that secures the position of the first support shaft relative to the second support shaft.

35. The apparatus of claim 34, further comprising a non-rigid coupling that couples the first support shaft to the first manipulating device.

36. The apparatus of claim 34, further comprising a non-rigid coupling that couples the second support shaft to the second manipulating device.

37. The apparatus of claim 34, wherein the first manipulating device is configured to bear a substantial amount of the weight of the organ and the second manipulating device is configured to substantially position the organ.

38. The apparatus of claim 34, wherein the first manipulating defines a first chamber and the second manipulating defines a second chamber.

39. The apparatus of claim 38, wherein the second support shaft is hollow and is in fluid communication with the second chamber.

40. The apparatus of claim 38 further comprising:
a first vacuum tube in fluid communication with the first chamber; and
a second vacuum tube in fluid communication with the second chamber.

41. The apparatus of claim 34 further comprising a positioning joint that couples the second support shaft to the second manipulating device.

42. The apparatus of claim 41, the positioning joint comprising:
a first hub; and
a second hub movably coupled to the first hub;
wherein at least one of the first hub and the second hub includes a restricting structure that limits the motion of the first hub relative to the second hub.

43. The apparatus of claim 34 wherein the structural connector comprises a first housing that receives the first support shaft and a second housing that receives the second support shaft.

44. The apparatus of claim 43, wherein the first housing and the second housing may be in one of an engaged position and a disengaged position, are positionable relative to one another when in the disengaged position and resist motion relative to one another when in the engaged position.

45. The apparatus of claim 44, wherein the structural connector further comprises a threaded connecting pin and a knob that receives the connecting pin, wherein the first housing and the second housing may be forced into one of the engaged position and the disengaged position by twisting the knob.

46. The apparatus of claim 44, wherein the first housing includes a first mating surface and the second housing includes a second mating surface, wherein the first mating surface contacts the second mating surface when the first housing and the second housing are in the engaged position, and wherein the first and second mating surfaces resist motion relative to one another when the first housing and the second housing are in the engaged position.

47. The apparatus of claim 34, wherein the first housing comprises an aperture to receive the first support shaft.

48. The apparatus of claim 34, wherein the second housing comprises an aperture to receive the second support shaft.

49. The apparatus of claim 48, wherein the second housing comprises a sleeve with an aperture that slidably receives the second support shaft.

50. The apparatus of claim 48, wherein the second support shaft includes a travel stop sized to be unable to pass through the aperture.

51. A method for manipulating an organ comprising:
engaging a first manipulating device with an apex of a heart to define a first chamber, at least a portion of the first manipulating device being compliant and adhesive to heart tissue;
engaging a second manipulating device with the heart at a site other than the apex to define a second chamber, at least a portion of the second manipulating device being compliant and adhesive to the heart tissue;
applying vacuum pressure to the first and second chambers such that a portion of each of the first and second manipulating devices deforms to substantially seal the first and second chambers against leakage;
substantially supporting the weight of the heart with the first manipulating device; and
positioning the heart with the second manipulating device.

52. The method of claim 51, further comprising securing the first and second manipulating devices in a substantially fixed position relative to one another.

53. The method of claim 52, wherein securing the first and second manipulating devices in a substantially fixed position relative to one another comprises:
positioning a first support shaft coupled to the first manipulating device in an adjustable structural connector;
positioning a second support shaft coupled to the second manipulating device in the adjustable structural connector; and
securing the position of the first and second support shafts with a securing member.

54. The method of claim 51, further comprising delivering vacuum pressure to the second manipulating device via the second support shaft.

55. A method comprising:
engaging a first manipulating device with an organ;
engaging a second manipulating device with the organ;
orienting a first support shaft into a position relative to a second support shaft, the first support shaft being coupled to the first manipulating device and the second support shaft being coupled to the second manipulating device;
securing the first support shaft into the position relative to the second support shaft;
substantially supporting the weight of the organ with the first manipulating device; and
positioning the organ with the second manipulating device.

56. The method of claim 55, wherein the first manipulating device defines a first chamber and the second manipulating device defines a second chamber, the method further comprising:
applying vacuum pressure to the first chamber to cause a portion of the first manipulating device to deform to substantially seal the first chamber against leakage; and
applying vacuum pressure to the second chamber to cause a portion of the second manipulating device to deform to substantially seal the second chamber against leakage.

57. The method of claim 55, wherein the organ is a heart.

58. A method comprising:
engaging a first manipulating device to an organ;
lifting the organ with the first manipulating device;
engaging a second manipulating device to the organ; and
positioning the organ with the second manipulating device.

59. The method of claim 58, further comprising securing the first and second manipulating devices in a substantially fixed position relative to one another.

60. The method of claim 59, wherein securing the first and second manipulating devices in a substantially fixed position relative to one another comprises:
positioning a first support shaft coupled to the first manipulating device in an adjustable structural connector;
positioning a second support shaft coupled to the second manipulating device in the adjustable structural connector; and
securing the position of the first and second support shafts with a securing member.
